SEQUENCE LISTING (14 sequences)

SEQ ID NO: 1 - DNA, 23 bp, Artificial Sequence
  Synthetic oligonucleotide
ttttgagttaggtgtgggatata

SEQ ID NO: 2 - DNA, 21 bp, Artificial Sequence
  Synthetic oligonucleotide
aaaatcaaaaaattccctttc

SEQ ID NO: 3 - DNA, 20 bp, Artificial Sequence
  Synthetic oligonucleotide
agttaggtgtgggatatagt

SEQ ID NO: 4 - DNA, 23 bp, Artificial Sequence
  Synthetic oligonucleotide
tttgattggttggttattttaga

SEQ ID NO: 5 - DNA, 21 bp, Artificial Sequence
  Synthetic oligonucleotide
aatatcctaatcactcccttc

SEQ ID NO: 6 - DNA, 19 bp, Artificial Sequence
  Synthetic oligonucleotide
ggttttataaatatagttg

SEQ ID NO: 7 - DNA, 22 bp, Artificial Sequence
  Synthetic oligonucleotide
tgaggctgttgtcatacttctc

SEQ ID NO: 8 - DNA, 16 bp, Artificial Sequence
  Synthetic oligonucleotide
cagccgagccacatcg

SEQ ID NO: 9 - DNA, 23 bp, Artificial Sequence
  Synthetic oligonucleotide
agaggagctcttggagacgacat

SEQ ID NO: 10 - DNA, 23 bp, Artificial Sequence
  Synthetic oligonucleotide
actcctgcttgttaattccgacc

SEQ ID NO: 11 - DNA, 23 bp, Artificial Sequence
  Synthetic oligonucleotide
aacctgtctgcctgtgaccaagt

SEQ ID NO: 12 - DNA, 25 bp, Artificial Sequence
  Synthetic nucleotide
acatcagcagcgttaacattttctg

SEQ ID NO: 13 - DNA, 20 bp, Artificial Sequence
  Synthetic oligonucleotide
cctgcagcacgttatcgact

SEQ ID NO: 14 - DNA, 18 bp, Artificial Sequence
  Synthetic oligonucleotide
ctcagcggcacagaatgc

(19) United States
(12) Patent Application Publication
(10) Pub. No.: US 2008/0234223 A1
(43) Pub. Date: Sep. 25, 2008

(54) N4 MODIFICATIONS OF PYRIMIDINE ANALOGS AND USES THEREOF

(75) Inventors: Allen S. Yang, Valencia, CA (US); Victor Marquez, Los Angeles, CA (US); Hyang-Min Byun, Glendale, CA (US); Jean-Pierre Issa, Bellaire, TX (US)

Correspondence Address: HOGAN & HARTSON;Suite 1400, 1999 Avenue of the Stars, Los Angeles, CA, 90067, US

(73) Assignee: UNIVERSITY OF SOUTHERN CALIFORNIA, Los Angeles, CA (US)

(21) Appl. No.: 11/929,556

(22) Filed: Oct. 30, 2007

Related U.S. Application Data
(60) Provisional application No. 60/855,220, filed on Oct. 30, 2006.
(60) Provisional application No. 60/943,172, filed on Jun. 11, 2007.

(51) Int. Cl. A61K 31/70; C12Q 1/02; C07D 403/00; C12N 5/06

(52) U.S. Cl. 514/49; 435/29; 544/296; 435/375

(57) ABSTRACT

The present invention relates to N4 carboxylester or ester derivatives of pyrimidine analogs with increased stability, solubility, and bioavailability. Also disclosed are methods of using these compounds for targeted drug delivery and combination therapy.

### RELATED APPLICATION

This application claims priority to U.S. Provisional Patent Application Ser. Nos. 60/855,220, filed on Oct. 30, 2006 and 60/943,172, filed on Jun. 11, 2007, the contents of which are incorporated herein by reference in their entirety.

### FIELD OF THE INVENTION

The present invention relates to N4 carboxylester or ester derivatives of pyrimidine analogs. These compounds have increased stability, solubility, and bioavailability, and can be used for targeted drug delivery and combination therapy.

### BACKGROUND OF THE INVENTION

5-methylcytosine comprises approximately 1% of the human genome, and arises from the post-replicative modification of cytosine by a family of methyltransferase enzymes (1). Humans are known to have three active enzymes, DNA methyltransferase (DNMT) 1, 3a, and 3b that transfer a methyl group from S-adenosylmethionine to the 5 position of cytosine in CpG dinucleotides (2). DNA methylation along with histones and histone modification control chromatin structure and gene expression. Abnormal DNA methylation patterns are commonly found in almost all cancer, and are associated with aberrant silencing of tumor suppressor genes (3, 4). In addition, some cancers appear to have phenotypes that drive aberrant DNA methylation, such as the CpG Island Methylator Phenotype (CIMP) (5).

Cytosine analogs are routinely used in the treatment of cancer as chemotherapeutic agents. One of the most important breakthroughs in the study of DNA methylation came with the discovery that 5-aza derivatives of cytosine, which have a nitrogen substitution in the 5-position of the ring, can inhibit DNA methylation (6). 5-azacytidine (azacitidine, Vidaza) is a ribonucleotide analog, and 5-aza-2′-deoxyazacytidine (decitabine, Dacogen), is a deoxyribonucleotide analog. These analogs once incorporated into the DNA in place of cytosine, can covalently trap DNA methyltransferase leading to inhibition of DNA methylation (7). These mechanistic inhibitors of DNA methylation are currently approved for use in myelodysplastic syndrome, and have clinical activity in other malignancies. Two other pyrimidine analogs, 5-fluorocytidine (8) and zebularine (9), are also mechanistic inhibitors of DNA methylation and are currently under clinical development.

Biologically it is clear that mechanistic inhibitors of DNA methylation work best at low doses with prolonged exposures. Jones and Taylor showed that muscle differentiation of 10T1/2 cells induced by azacitidine was due to inhibition of DNA methylation and optimal at lower concentrations of the drug (10). Clearly lower doses of azacytidine or decitabine are more effective at inhibiting DNA methylation in vitro and in vivo (11, 12). At higher doses the drugs are less effective at inhibiting DNA methylation and cause cytotoxicity. Consistent with this biological observation, Issa and colleagues have shown that clinically these drugs are most effective at a dose of 15 mg/m²/day for 10 days, which is approximately 1/20 the maximum tolerated dose with 11 of 18 patients with refractory leukemia responding (12). Corresponding studies on DNA methylation changes in the peripheral blood of these patients showed that this dose, 15 mg/m²/day was the optimal biological dose for inhibiting DNA methylation (13).

In addition to low doses, prolonged exposure to these drugs is important for their effectiveness. Nucleotide analogs that require incorporation into DNA are S-phase specific and therefore azacytosine derivatives are also more effective at inhibiting DNA methylation with prolonged exposure (14). Unfortunately, azacytosine nucleotides are not stable in aqueous solution. The azacytosine ring of both 5-azacytidine and 5-aza-2′-deoxyazacytidine can undergo hydrolysis to an inactive form (15). The half-life of 5-azacytidine is 1.5+/−2.3 hours (16) and the half-life of 5-aza-2′-deoxyazacytidine is 15 to 25 minutes in aqueous solution (17). The need for prolonged administration, ideally continuous infusion, is impractical due to the aqueous instability of the drug. Thus, the drugs require frequent administration and immediate use of the drug once reconstituted from the lyophilized powder form. Compounding the aqueous instability is the fact that cytidine deaminase rapidly metabolizes both azacitidine and decitabine after administration. The in vivo plasma half-life of azacitidine and decitabine are only 41 and 7 minutes respectively due to rapid deamination to azauridine by plasma cytidine deaminase (18, 19). Thus, there is a need to make drugs that are more stable and are better able to withstand the rapid metabolism by cytidine deaminase. The ability to stabilize the pyrimidine ring in a prodrug will also help make the drug orally bioavailable.

Patients treated with decitabine show clear decreases in global DNA methylation, but DNA methylation levels quickly return to baseline levels well before the next course and usually within days of stopping drug administration (13). Development of an oral mechanistic inhibitor of DNA methylation that could be given continuously would provide a convenient route of drug administration that could improve the clinical ability to inhibit DNA methylation and clinical efficacy. Zebularine, another pyrimidine analog, is more stable than azacytosine pyrimidine analogs, and is potentially orally bioavailable (20). However, zebularine is not efficiently metabolized to the triphosphate form and therefore is 100 times less potent than decitabine at inhibiting DNA methylation (20).

### SUMMARY OF THE INVENTION

In one aspect, the invention features a method of reducing nucleic acid methylation in a cell. The method comprises identifying a cell in which the level of nucleic acid methylation is higher than a control level and contacting the cell with a compound, thereby reducing nucleic acid methylation in the cell. The compound contains an epigenetic agent, a cell targeting agent, or a carbon chain or ring containing 2-10 carbons attached to a pyrimidine analog that inhibits nucleic acid methylation through an N4 carboxylester or ester bond.

Examples of pyrimidine analogs include, but are not limited to, 5-azacytidine, 5-aza-2′-deoxycytidine, and 5-fluorocytidine. Examples of epigenetic agents include, but are not limited to, histone deacetylase inhibitors, histone methyltransferase inhibitors, methyl-binding protein inhibitors, and SIRT (sirtuin class of human protein deacetylases) inhibitors.

In some embodiments, the compound may be NPEOC-DAC, the cell may express carboxylesterase 1 (CES 1), and the cell may be a liver cancer cell. In some embodiments, the cell-targeting agent may be acetylcholine, the cell may express acetylcholinesterase, and the cell may be a cell of the central nervous system.

In another aspect, the invention features a composition comprising a pharmaceutically acceptable carrier and a compound in which an epigenetic agent, a cell targeting agent, or a carbon chain or ring containing 2-10 carbons is attached to a pyrimidine analog that inhibits nucleic acid methylation through an N4 carboxylester or ester bond. For example, the compound may be NPEOC-DAC, and the pharmaceutically acceptable carrier may be a hydrophobic solvent, a polar solvent, or a hydrophobic-polar solvent, such as dimethyl sulfoxide (DMSO).

The invention further provides a compound comprising a therapeutic agent or a cell-targeting agent attached to a pyrimidine analog through an N4 carboxylester or ester bond. In some embodiments, the therapeutic agent may be an epigenetic agent. In some embodiments, the pyrimidine analog may be 5-azacytidine, 5-aza-2′-deoxycytidine, 5-fluorocytidine, or cytosine arabinoside.

In particular, the invention provides the compounds having the following structures:

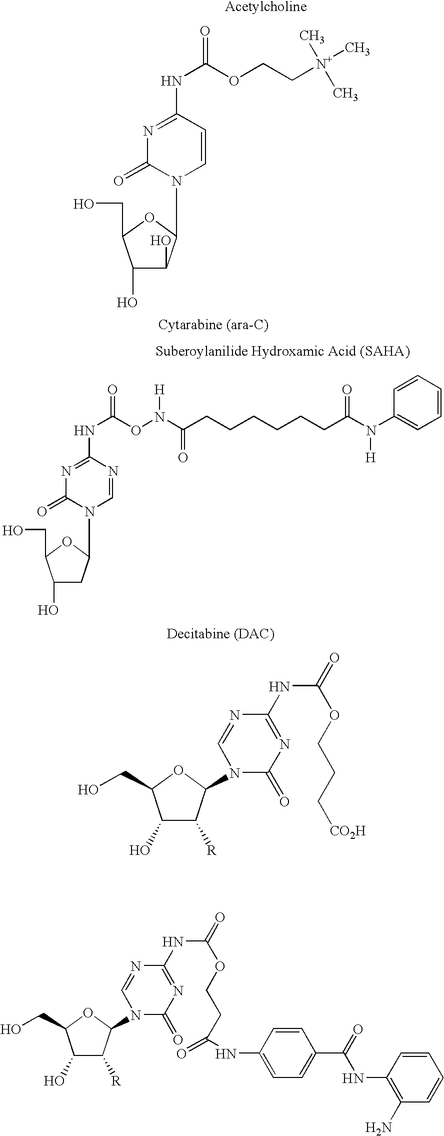

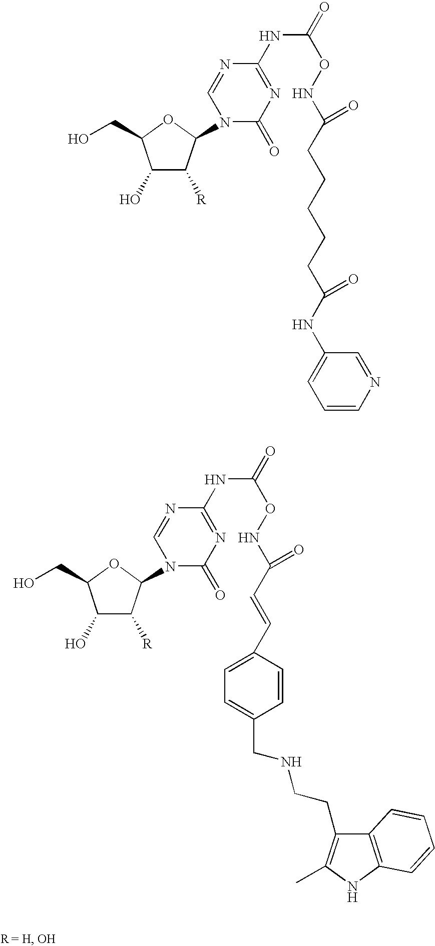

These compounds have increased stability, solubility, and bioavailability, and can be used for targeted drug delivery and combination therapy.

Also within the invention is a method of modulating the biological activity of a cell. The method comprises contacting a cell with a compound of the invention, thereby modulating the biological activity of the cell. For example, the compound may be used to treat a cancer cell when the compound contains a cancer therapy agent.

The above-mentioned and other features of this invention and the manner of obtaining and using them will become more apparent, and will be best understood, by reference to the following description, taken in conjunction with the accompanying drawings. The drawings depict only typical embodiments of the invention and do not therefore limit its scope.

### BRIEF DESCRIPTION OF THE FIGURES

FIG. 1 shows the structures of (A) 5-aza-2′-deoxycytidine (decitabine, DAC) and (B) N4 2-(p-nitrophenyl)ethoxycarbonyl 5-aza-2′-deoxycytidine (NPEOC-DAC). The grey oval is the NPEOC region.

FIG. 2 shows DNA methylation changes induced by DAC and NPEOC-DAC. Various cell lines were treated with either 10 μM DAC or 10 μM NPEOC-DAC for three days. Global DNA methylation was quantified using bisulfite-PCR pyrosequencing of the LINE-1 repetitive element. All experiments were performed in triplicate and error bars represent standard deviation.

FIG. 3 shows that ID4 methylation is decreased by DAC and NPEOC-DAC. Cell lines were treated with either 10 μM DAC or 10 μM NPEOC-DAC for 3 days. DNA methylation of the ID4 gene was quantified using bisulfite-PCR pyrosequencing. All experiments were performed in triplicate and error bars represent standard deviation.

FIG. 4 shows that DAC and NPEOC-DAC reactivate the expression of ID4. HepG2 cells were treated with DAC (A) or NPEOC-DAC (B) at the concentrations indicated (μM). Total RNA was isolated after 3 days of treatment and mRNA expression of ID4 was measured by RT-PCR. Untreated HepG2 cells had no detectable ID4 expression, but treatment by either DAC (A) or NPEOC-DAC (13) lead to expression of ID4.

FIG. 5 shows carboxylesterase expression in various cell lines. Reverse transcriptase multiplex PCR of carboxylesterase 1 and carboxylesterase 2 was performed on cDNA from the cell lines studied. Expression of CES1, the most abundantly expressed carboxylesterase, was limited to the liver cancer cell lines, Hep3B and HepG2.

FIG. 6 shows that LINE-1 DNA methylation changes at various concentrations of DAC and NPEOC-DAC. T24, a bladder cancer cell line, and HepG2, a liver cancer cell line, were treated for 3 days with various concentrations of either DAC (upper panel) or NPEOC-DAC (lower panel).

FIG. 7 shows the time course of DNA methylation changes. T24 and HepG2 cell lines were treated continually with either DAC (10 μM) or NPEOC-DAC (10 μM). DNA methylation of LINE-1 was assessed on days 1, 3, 7, and 14.

FIG. 8 shows that NDGA inhibits the ability of NPEOC-DAC to inhibit DNA methylation. HepG2 cells were treated with either DAC (1 μM) or NPEOC-DAC (100 μM) in the presence or absence of NDGA, a known inhibitor of carboxylesterase enzyme.

FIG. 9 shows results of multiplex reverse transcription PCR using cDNA from various human tissues. CES1 and CES2 expression levels vary from tissue to tissue with GAPDH as a quantitative control for starting cDNA template.

FIG. 10 shows the structure of N4-acetylcholine cytosine arabinoside. Cytosine arabinoside (ara-C, cytarabine) has been modified in the N4 position with acetylcholine attached via an ester bond. This molecule would be targeted specifically to tissues that express acetylcholinesterase, such as the central nervous system.

FIG. 11 shows the structure of N4-suberoylanilide hydroxamic acid-5-aza-2′-deoxycytidine. The N4 position of 5-aza-2′-deoxycytidine has been modified to add suberoylanilide hydroxamic (SARA) via a carboxylester bond. This molecule will have the stability and oral bioavailability of NPEOC-DAC, but will release decitabine, a DNA methylation inhibitor, and SARA, a histone deacetylase inhibitor.

FIG. 12 shows additional structures of the compounds of the invention.

### DETAILED DESCRIPTION OF THE INVENTION

It is an object of the present invention to use N4 carboxylester or ester derivatives of 5-azacytidine or other azacytosine pyrimidine analogs or other nucleotide analogs to deliver mechanistic inhibitors of DNA methylation orally. The modification of 5-aza-2′-deoxycytidine at the N4 position was first carried out to stabilize the azacytosine ring during production of a phosphoramidate version of azacytidine that would be chemically stable enough to be incorporated into a synthetic oligonucleotide. The inventors demonstrate that one intermediate of the NPEOC protected phosphoramidite of azacytidine, NPEOC-DAC, is able to specifically inhibit DNA methylation after conversion of NPEOC-DAC into DAC by the cleavage of the N4 nitrophenyl group by a carboxylesterase enzyme. The inventors have found NPEOC-DAC, unlike DAC, to have very low solubility in water and consequently dissolved NPEOC-DAC in DMSO. The increased chemical stability of NPEOC-DAC and the hydrophobicity of the compound should provide very favorable human pharmacokinetics including oral bioavailability. Therefore, one can use NPEOC-DAC or some other N4 derivative of 5-aza-2′-deoxycytidine or 5-azacytidine to administer these drugs orally, have them survive the acidic environment of the stomach, and then be metabolized to an active pyrimidine analog by first pass metabolism of the liver.

It is another object of the present invention to use N4 carboxylester or ester derivatives of azacytosine pyrimidine analogs or other nucleotide analogs to increase their aqueous stability and avoid deamination by cytidine deaminase. The increased in vitro and in vivo stability of these derivatives should provide ease of prolonged administration and prolonged bioavailability, which in turn should provide an enhanced ability to inhibit DNA methylation and increased therapeutic activity.

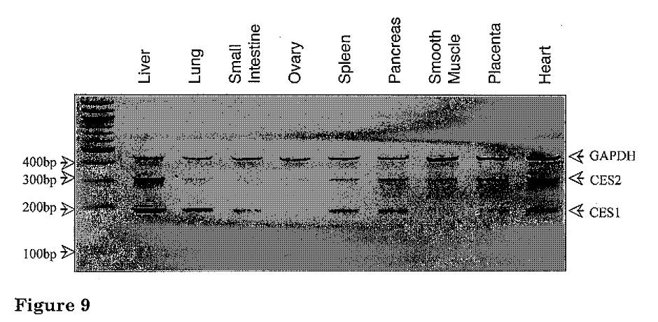

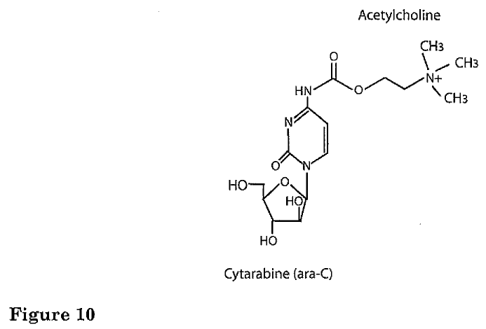

It is another object of the present invention to use N4 carboxylester or ester derivatives of azacytosine pyrimidine analogs and other nucleotide analogs for specific tissue or cell targeting. As an example of this embodiment, the inventors present NPEOC-DAC that can be cleaved by carboxylesterase. Carboxylesterase consists of a family of enzymes that have different specificities for different carboxylesterase molecules. For example, CES3 is known to be expressed specifically in the brain. The expression pattern of carboxylesterase varies from tissue to tissue (FIG. 9), which can be exploited pharmacologically. One could modify the N4 carboxylester bond and side group to target specific members of the carboxylesterase enzyme family, and therefore, target activation and release of the active drug in specific tissues or organs. Conversely, the N4 group could be modified to avoid certain tissues, such as the bone marrow, and therefore, avoid unwanted toxicity such as myelosuppression. This would allow organ specific targeting of epigenetic therapy or nucleotide analog therapy. One of ordinary skill in the art would realize that this embodiment can easily be applied to other nucleotide analogs as well. For example, this embodiment could be applied using cytosine arabinoside (ara-C, cytarabine) with an acetylcholine group attached via an ester bond. This example could be activated specifically by acetylcholinesterase to deliver ara-C to the central nervous system (FIG. 10).

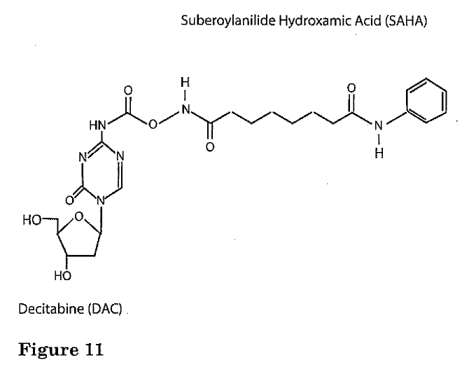

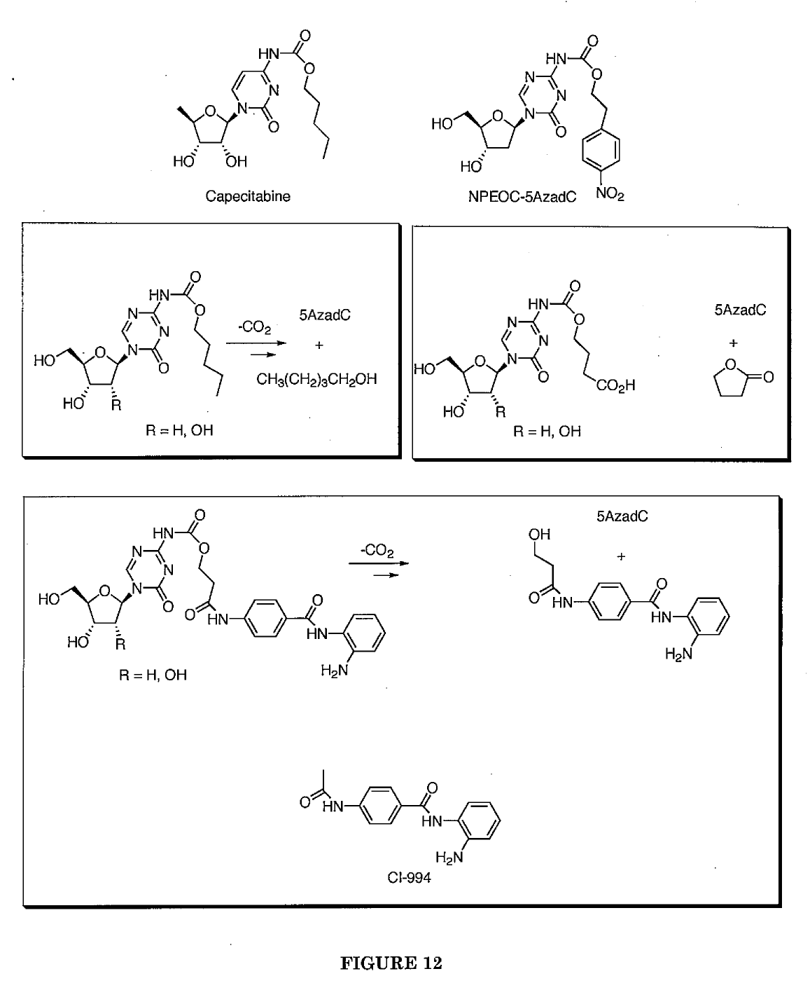

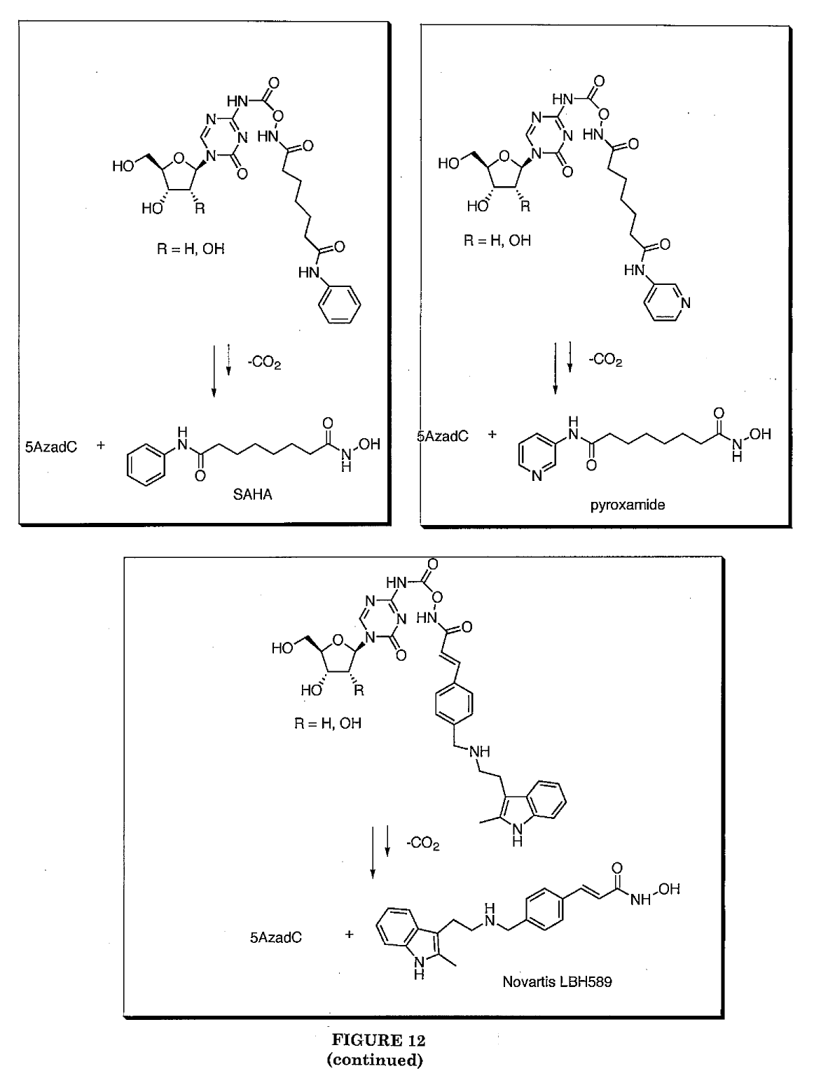

Yet another object of the present invention is to use N4 carboxylester or ester derivatives of azacytosine pyrimidine analogs and other nucleotide analogs to deliver combination therapies. Drugs can be modified to make prodrugs that combine two active drugs via a carboxylester or ester bond. This embodiment allows the cleavage of the carboxylester or ester bond to release two active drugs from a single prodrug. As an example this embodiment, the inventors present N4-suberoylanilide hydroxamic acid-5-aza-2′-deoxycytidine. DNA methylation inhibitors can be combined with other epigenetic agents such as histone deacetylase inhibitors. Some of these inhibitors such as valproic acid, phenylbutyrate, and suberoylanilide hydroxamic acid (SAHA) consist of carbon chains that could be attached to decitabine, azacitidine, or other nucleotide analog at the N4 position with a carboxylester bond (FIG. 11). Addition of another active agent to the N4 position of DAC would then lead to a two-sided drug where cleavage of the carboxylester bond could release two active agents. Therefore, as can be recognized by a person of ordinary skill in the art, this prodrug system could not only be used for the targeted delivery of a DNA methylation inhibitor, but also for the delivery of a combination of epigenetic therapies or other therapies depending on the group added for the N4 modification.

Accordingly, the invention provides a method of reducing nucleic acid methylation in a cell in vitro and in vivo. A cell to be treated has a nucleic acid methylation level higher than a control level. Such a cell may be identified by measuring the nucleic acid methylation level and comparing it to a control level, e.g., the level of nucleic acid methylation in a normal cell. Nucleic acid methylation in a cell can be determined by any of the methods known in the art, for example, methylation-specific PCR (MSP), bisulfite sequencing, or pyrosequencing.

MSP is a technique whereby DNA is amplified by PCR dependent upon the methylation state of the DNA. See, e.g., U.S. Pat. No. 6,017,704. Determination of the methylation state of a nucleic acid includes amplifying the nucleic acid by means of oligonucleotide primers that distinguish between methylated and unmethylated nucleic acids. MSP can rapidly assess the methylation status of virtually any group of CpG sites within a CpG island, independent of the use of methylation-sensitive restriction enzymes. This assay entails initial modification of DNA by sodium bisulfite, converting all unmethylated, but not methylated, cytosines to uracils, and subsequent amplification with primers specific for methylated versus unmethylated DNA. MSP requires only small quantities of DNA, is sensitive to 0.1% methylated alleles of a given CpG island locus, and can be performed on DNA extracted from cells and tissue samples. MSP products can be detected by gel electrophoresis, CAE (capillary array electrophoresis), or real-time quantitative PCR.

Bisulfite sequencing is widely used to detect 5-MeC (5-methylcytosine) in DNA, and provides a reliable way of detecting any methylated cytosine at single-molecule resolution in any sequence context. The process of bisulfite treatment exploits the different sensitivity of cytosine and 5-MeC to deamination by bisulfite under acidic conditions, in which cytosine undergoes conversion to uracil while 5-MeC remains unreactive.

Pyrosequencing is a method of DNA sequencing based on the “sequencing by synthesis” principle. The method involves a chemical light-producing enzymatic reaction, which is triggered when a molecular recognition event occurs. Essentially, the method allows sequencing of a single strand of DNA by synthesizing the complementary strand along it. Each time a nucleotide, A, C, G, or T is incorporated into the growing chain, a cascade of enzymatic reactions is triggered which causes a light signal. In pyrosequencing, a single-stranded DNA template is hybridized to a sequencing primer and incubated with the enzymes DNA polymerase, ATP sulfurylase, luciferase, and apyrase, and with the substrates adenosine 5′ phosphosulfate (APS) and luciferin. The addition of one of the four deoxynucleotide triphosphates (dNTPs) initiates the second step. DNA polymerase incorporates the correct, complementary dNTPs onto the template. This incorporation releases pyrophosphate (PPi) stoichiometrically. ATP sulfurylase quantitatively converts PPi to ATP in the presence of adenosine 5′-phosphosulfate. This ATP acts as fuel to the luciferase-mediated conversion of luciferin to oxyluciferin that generates visible light in amounts that are proportional to the amount of ATP. The light produced in the luciferase-catalyzed reaction is detected by a charge coupled device (CCD) camera and this can be analyzed in a program. Each light signal is proportional to the number of nucleotides incorporated. Unincorporated nucleotides and ATP are degraded by the apyrase, and the reaction can restart with another nucleotide.

The identified cell is then contacted with a compound for reducing nucleic acid methylation in the cell. The compound contains an epigenetic agent, a cell targeting agent, or a carbon chain or ring containing 2-10 carbons attached to a pyrimidine analog that inhibits nucleic acid methylation through an N4 carboxylester or ester bond. Such a compound may be obtained using any of the synthesis methods well known to a skilled artisan. See, for example, Garcia et al. (2001) Antisense Nucleic Acid Drug Dev 11, 369 for synthesis of NPEOC-DAC.

An epigenetic agent refers to a compound that regulates gene expression without changing the nucleotide sequence of the gene. Two key areas of epigenetic control are chromatin remodeling and DNA methylation. Examples of epigenetic agents include, but are not limited to, histone deacetylase inhibitors, histone methyltransferase inhibitors, methyl-binding protein inhibitors, and SIRT inhibitors. Epigenetic agents are known in the art. See, for example, Marks et al. (2000) Journal of the National Cancer Institute 92:1210-1216; Tan et al. (2007) Genes & Dev 21:1050-1063; and Yoo and Jones (2006) Nat Rev Drug Discov. 5(1):37-50.

A cell targeting agent is a molecule that directs a compound to a specific type of cell or tissue where the active component of the compound is released and becomes functional. For example, CES1 cleaves NPEOC-DAC to release DAC. This compound is thus useful for targeting cells and tissues (e.g., liver cancer cells) where CES1 is expressed. Similarly, acetylcholine may be attached to a nucleotide analog through an ester bond. This compound may be used to target cells and tissues (e.g., the central nervous system) where acetylcholinesterase is expressed.

A carbon chain or ring containing 2-10 carbons may be used to increase the stability, solubility, and bioavailability of a pyrimidine analog that inhibits nucleic acid methylation. For example, the carbon chain may contain a hydrophobic group.

A pyrimidine analog is a chemical structure that can be incorporated into a nucleic acid in place of cytosine, thymidine, or uracil. Some of the pyrimidine analogs such as 5-azacytidine, 5-aza-2′-deoxycytidine, and 5-fluorocytidine are known to have the ability to inhibit nucleic acid methylation. An example of a pyrimidine analog that does not inhibit nucleic acid methylation is cytosine arabinoside.

The invention also provides a composition comprising a pharmaceutically acceptable carrier, such as a buffer, solvent, and/or excipient, and a compound in which an epigenetic agent, a cell targeting agent, or a carbon chain or ring containing 2-10 carbons is attached to a pyrimidine analog that inhibits nucleic acid methylation through an N4 carboxylester or ester bond. For example, a hydrophobic solvent, a polar solvent, or a hydrophobic-polar solvent, such as DMSO, may be used to dissolve the compound. In one embodiment, the compound and the solvent may be separately kept. In another embodiment, the compound may be dissolved in the solvent.

Furthermore, the invention provides a compound comprising a therapeutic agent or a cell-targeting agent attached to a pyrimidine analog through an N4 carboxylester or ester bond. A therapeutic agent refers to any chemical structure that is useful for treating diseases or helping healing take place. For example, a therapeutic agent may be an epigenetic agent. The pyrimidine analog may be 5-azacytidine, 5-aza-2′-deoxycytidine, 5-fluorocytidine, or cytosine arabinoside. These compounds may be obtained by chemical synthesis using methods well known in the art, for example, those similar to what is described in Garcia et al. (2001) Antisense Nucleic Acid Drug Dev 11, 369. Such compounds have increased stability, solubility, and bioavailability, and can be used for targeted drug delivery and combination therapy.

A compound of the invention may be admixed with a pharmaceutically acceptable carrier to form a pharmaceutical composition. “Pharmaceutically acceptable carriers” include solvents, dispersion media, coatings, antibacterial and antifungal agents, isotonic and absorption delaying agents, and the like, compatible with pharmaceutical administration.

A compound or composition of the invention may be formulated to be compatible with its intended route of administration. See, e.g., U.S. Pat. No. 6,756,196. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates; and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Formulations suitable for injectable use include sterile aqueous solutions (where water soluble) or dispersions and sterile powders for the extemporaneous preparation of sterile injectable solutions or dispersion. For intravenous administration, suitable carriers include physiological saline, sterile water, Cremophor EL™ (BASF, Parsippany, N.J.), or phosphate buffered saline (PBS). In all cases, the formulation must be sterile and should be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, polyol (e.g., glycerol, propylene glycol, and liquid polyetheylene glycol), and suitable mixtures thereof. The proper fluidity can be maintained, for example, by the use of a coating such as lecithin, by the maintenance of the required particle size in the case of dispersion, and by the use of surfactants. Prevention of the action of microorganisms can be achieved by various antibacterial and antifungal agents, for example, parabens, chlorobutanol, phenol, ascorbic acid, and thimerosal. In many cases, it will be preferable to include isotonic agents, for example, sugars, polyalcohols such as manitol, sorbitol, and sodium chloride in the formulation. Prolonged absorption of the injectable formulations can be brought about by including in the formulation an agent which delays absorption, for example, aluminum monostearate and gelatin.

Sterile injectable solutions can be prepared by incorporating the compounds or compositions in the required amounts in an appropriate solvent with one or a combination of ingredients enumerated above, as required, followed by filtered sterilization. Generally, dispersions are prepared by incorporating the compounds or compositions into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral formulations generally include an inert diluent or an edible carrier. For the purpose of oral therapeutic administration, the compounds can be incorporated with excipients and used in the form of tablets, troches, or capsules, e.g., gelatin capsules. Oral formulations can also be prepared using a fluid carrier for use as a mouthwash. Pharmaceutically compatible binding agents, and/or adjuvant materials can be included as part of the formulation. The tablets, pills, capsules, troches, and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the formulations are delivered in the form of an aerosol spray from pressured container or dispenser which contains a suitable propellant, e.g., a gas such as carbon dioxide and a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the compounds or compositions are formulated into ointments, salves, gels, or creams as generally known in the art.

The formulations of the invention can also be prepared in the form of suppositories (e.g., with conventional suppository bases such as cocoa butter and other glycerides) or retention enemas for rectal delivery.

In one embodiment, the formulations are prepared with carriers that will protect the compounds or compositions against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is advantageous to prepare oral or parenteral formulations in dosage unit form for ease of administration and uniformity of dosage. “Dosage unit form,” as used herein, refers to physically discrete units suited as unitary dosages for a subject to be treated; each unit containing a predetermined quantity of active compound or composition calculated to produce the desired therapeutic effect in association with the required pharmaceutical carrier.

The formulations of the invention can be included in a container, pack, or dispenser together with instructions for administration to form packaged products. Other active compounds can also be incorporated into the formulations.

In addition, the invention provides a method of modulating the biological activity of a cell such as DNA methylation, gene expression, and protein activity by contacting a cell with a compound of the invention. For example, when a compound contains a cancer therapy agent, the compound may be used to treat a cancer cell.

Moreover, the invention provides both prophylactic and therapeutic methods of treating a subject in need thereof by administering to the subject an effective amount of a compound or composition of the invention.

“Subject,” as used herein, refers to a human or animal, including all vertebrates, e.g., mammals, such as primates (particularly higher primates), sheep, dog, rodents (e.g., mouse or rat), guinea pig, goat, pig, cat, rabbit, cow; and non-mammals, such as chicken, amphibians, reptiles, etc. In a preferred embodiment, the subject is a human. In another embodiment, the subject is an animal.

A subject to be treated may be identified, e.g., using diagnostic methods known in the art, as being suffering from or at risk for developing a disease or condition. The subject may be identified in the judgment of a subject or a health care professional, and can be subjective (e.g., opinion) or objective (e.g., measurable by a test or diagnostic method). For example, the DNA methylation level in a test subject may be measured as described above and compared with the level in a healthy subject. If the DNA methylation level in the test subject is higher than the level in the healthy subject, the test subject may be identified as a candidate for treatment with a compound or composition of the invention that inhibits DNA methylation.

As used herein, the term “treatment” is defined as the application or administration of a therapeutic agent to a subject, or application or administration of a therapeutic agent to an isolated tissue or cell line from a subject, who has a disease, a symptom of a disease, or a predisposition toward a disease, with the purpose to cure, heal, alleviate, relieve, alter, remedy, ameliorate, improve, or affect the disease, the symptoms of the disease, or the predisposition toward the disease.

An “effective amount” is an amount of a therapeutic agent that is capable of producing a medically desirable result as delineated herein in a treated subject. The medically desirable result may be objective (i.e., measurable by some test) or subjective (i.e., subject gives an indication of or feels an effect).

Toxicity and therapeutic efficacy of a compound or composition of the invention can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index, and can be expressed as the ratio LD₅₀/ED₅₀. Compounds or compositions which exhibit high therapeutic indices are preferred. While compounds or compositions that exhibit toxic side effects may be used, care should be taken to design a delivery system that targets such compounds or compositions to the site of affected tissue in order to minimize potential damage to uninfected cells and, thereby, reduce side effects.

The data obtained from the cell culture assays and animal studies can be used in formulating a range of dosage for use in humans. The dosage of the compounds lies preferably within a range of circulating concentrations that include the ED₅₀ with little or no toxicity. The dosage may vary within this range depending upon the dosage form employed and the route of administration utilized. For any compound or composition to be used in a method of the invention, the therapeutically effective dose can be estimated initially from cell culture assays. A dose may be formulated in animal models to achieve a circulating plasma concentration range that includes the IC₅₀ (i.e., the concentration of a compound which achieves a half-maximal inhibition of symptoms) as determined in cell culture. Such information can be used to more accurately determine useful doses in humans. Levels in plasma may be measured, for example, by high performance liquid chromatography.

A therapeutically effective amount of the compounds or compositions (i.e., an effective dosage) may range from, e.g., about 1 microgram per kilogram to about 500 milligrams per kilogram, about 100 micrograms per kilogram to about 5 milligrams per kilogram, or about 1 microgram per kilogram to about 50 micrograms per kilogram. The compounds can be administered, e.g., one time per week for between about 1 to 10 weeks, preferably between 2 to 8 weeks, more preferably between about 3 to 7 weeks, and even more preferably for about 4, 5, or 6 weeks. In subjects suffering from chronic diseases, such as arthritis or osteoporosis, life-long treatment may be necessary, for example, one time every day or preferably one time per week. It is furthermore understood that appropriate doses of a compound depend upon the potency of the compound or composition. When one or more of these compounds or compositions are to be administered to a subject (e.g., an animal or a human), a physician, veterinarian, or researcher may, for example, prescribe a relatively low dose at first, subsequently increasing the dose until an appropriate response is obtained. In addition, it is understood that the specific dose level for any particular subject will depend upon a variety of factors including the activity of the specific compound or composition employed, the age, body weight, general health, gender, and diet of the subject, the time of administration, the route of administration, the rate of excretion, any drug combination, the severity of the disease or disorder, previous treatments, and other diseases present. Moreover, treatment of a subject with a therapeutically effective amount of the compounds or compositions can include a single treatment or, preferably, can include a series of treatments.

A compound or composition of the invention may be used alone, or in combination with other therapeutic agents. The compound or composition of the invention and the other therapeutic agents may be administered simultaneously or sequentially, as mixed or individual dosages. A compound or composition of the invention may be administered parenterally, intradermally, subcutaneously, orally, transdermally, transmucosally, or rectally.

The following examples are intended to illustrate, but not to limit, the scope of the invention. While such examples are typical of those that might be used, other procedures known to those skilled in the art may alternatively be utilized. Indeed, those of ordinary skill in the art can readily envision and produce further embodiments, based on the teachings herein, without undue experimentation.

### EXAMPLES

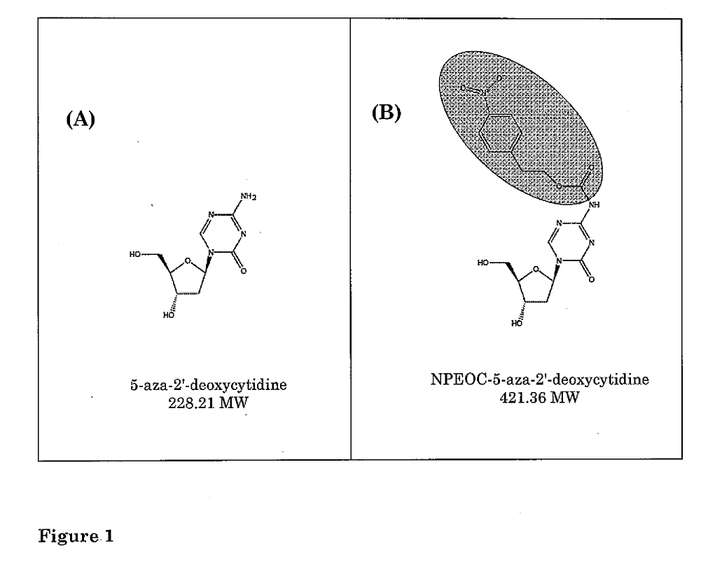

In order to study 5-aza-derivatives of cytosine, azacytosine has been chemically incorporated into an oligonucleotide (21). In order to protect the azacytosine ring during oligonucleotide synthesis, a 2-(p-nitrophenyl)ethoxycarbonyl (NPEOC) protecting group was added to the N4 position of the azacytosine ring, creating N4-NPEOC-DAC (FIG. 1). The NPEOC group was then removed chemically using 1,8-diazabiciclo [5.4.0]undec-7-ene (DBU) after synthesis of the azacytidine containing oligonucleotide. The inventors believed that N4-NPEOC DAC might also inhibit DNA methylation in vivo. Carboxylesterases consist of a family of enzymes that hydrolyze ester and carboxylester bonds. These enzymes have a broad specificity and are involved in the metabolism of xeonobiotics (pesticides, CPT-11, nerve gases, heroin and other drugs). Specific carboxylesterases subtypes are variably expressed in different human tissues (22). While not wanting to be bound by the theory, the inventors believed that carboxylesterase enzymes could remove the NPEOC protecting group of NPEOC-DAC, and result in the direct release of 5-aza-2′-deoxycytidine in vivo. This is analogous to the prodrug capecitabine (Xeloda) (22), 5′-deoxy-5-fluoro-N-[(pentyloxy)carbonyl]-cytidine (23), which is a pyrimidine analog with the addition of a carbon chain on the N4 position via a carboxylester bond. Capecitabine can be converted to 5′-deoxy-5-fluorocytidine by carboxylesterase in the liver and then is further metabolized to 5-fluorouracil, the active chemotherapy agent (22). This drug is currently clinically used as an oral form of 5-fluorouracil in gastrointestinal and breast cancers (24, 25).

The present invention demonstrates that 2′-deoxy-N4-[2-(4-nitrophenyl)ethoxycarbonyl]-5-azacytidine (NPEOC-DAC) can be activated to produce 5-aza-2′-deoxycytidine. This prodrug can inhibit global and gene specific DNA methylation like other azacytosine analogs, but this activity is limited to cells that express carboxylesterase 1. The present invention further demonstrates that NPEOC-DAC can reactivate ID4 expression, a tumor suppressor gene frequently hypermethylated in cancer.

### Material and Methods

### Cell Lines

T24 cells (urinary bladder transitional cell carcinoma), MCF7 (breast adenocarcinoma), HepG2 cells (hepatocellular carcinoma), Hep3B (hepatocellular carcinoma), were obtained from American Type Culture Collection (Manassas, Va.). T24 were cultured in McCoy's 5A medium supplemented with 10% fetal bovine serum. Other cells were cultured in DMEM medium plus 10% fetal bovine serum. All cells were grown in a humidified 37° C. incubator containing 5% CO₂.

### Nucleic Acid Isolation

Genomic DNA was isolated by standard proteinase K digestion and phenol-chloroform extraction (26). Total RNA was collected and extracted from cultured cells with the RNeasy Protect minikit (Qiagen, Valencia, Calif.) according to the manufacturer's protocol.

### Drug Treatments

Cells were seeded at 2×10⁴ cells per well in a 12-well dish 24 hours prior to treatments. Cells were treated with 5-Aza-CdR (Sigma-Aldrich Chemical Company, St. Louis, Mo.), NPEOC-DAC at the indicated concentrations. 5-aza-2′-deoxyazacytidine was dissolved in distilled water and NPEOC-DAC was dissolved in dimethyl sulfoxide (DMSO). Untreated controls of the DMSO solvent alone were also included. Cells were treated at the concentrations indicated for each experiment. Cells were collected after the number of days indicated in individual experiments. For subsequent methylation, genomic DNA was extracted using standard phenol/chloroform extraction methods, as described previously (27). Each experiment was done in triplicate and standard deviations are indicated by error bars.

### Bisulfite Treatment

Bisulfite modification of genomic DNA has been described previously (28). In brief, 1.5 μg of DNA was denatured in 50 μl of 0.2 M NaOH for 10 min at 37° C. Then, 30 μl of freshly prepared 10 mM hydroquinone (Sigma) and 520 μl of 3 M sodium bisulfite (Sigma) at pH 5.0 were added and mixed. The samples were overlaid with mineral oil to prevent evaporation and incubated at 50° C. for 16 h. The bisulfite-treated DNA was isolated using Wizard DNA Clean-Up System (Promega). The DNA was eluted by 50 microliters of warm water and 5.5 microliters of 3 M NaOH were added for 5 min. The DNA was ethanol precipitated with glycogen as a carrier and resuspended in 20 microliters of water. Bisulfite treated DNA was stored at −20° C. until ready for use.

### Reverse Transcription and Multiplex Polymerase Chain Reaction Analysis

Total RNA (5 micrograms) extracted from cultured cells was reverse transcribed with Moloney murine leukemia virus reverse transcriptase (Invitrogen, Carlsbad, Calif.) and random hexamers (NEB) in a total volume of 20 microliters. The reverse transcription was performed according to the manufacturer's recommended protocol. To identify expression level of carboxylesterase enzyme, cDNA samples were amplified in a multiplex PCR reaction with glyceraldehyde-3-phosphate dehydrogenase (GAPDH), CES1, and CES2 primers. The gene specific primers were designed to have a similar Tₘ so that they have similar amplification kinetics by combination with 3 different pair of primers. For ID4 expression, cDNA samples were amplified with ID4 primers. Primer sequences are shown in Table 1.

TABLE 1

Primers used in quantitative pyrosequencing
assays and RT-PCR

| | | | |
|---|---|---|---|
| | | | Optimal |
| | | GenBank | annealing |
| Primer | Sequence (5′ to 3′) | accession # | temperature |

For bisulfite pyrosequencing

| | | | |
|---|---|---|---|
| LINE-F | (Sense) | X58075 | 53° C. |
| | TTTTGAGTTAGGTGTGGGA | | |
| | TATA | | |
| LINE-R | (Antisense)biotin- | | |
| | AAAATCAAAAAATTCCCTT | | |
| | TC | | |
| LINE-SP | (Sense) | | |
| | AGTTAGGTGTGGGATAT | | |
| | AGT | | |
| ID4-F | (Sense) | BC014941 | 55° C. |
| | TTTGATTGGTTGGTTATTT | | |
| | TAGA | | |
| ID4-R | (Antisense) biotin- | | |
| | AATATCCTAATCACTCCCT | | |
| | TC | | |
| ID4-SP | (Sense) | | |
| | GGTTTTATAAATATAGTTG | | |

For expression

| | | | |
|---|---|---|---|
| GAPDH-F | (Sense) | AB062273 | 63° C. |
| | TGAGGCTGTTGTCATACTT | | |
| | CTC | | |
| GAPDH-R | (Antisense) | | |
| | CAGCCGAGCCACATCG | | |
| CES1-F | (Sense) | AB119996 | 63° C. |
| | AGAGGAGCTCTTGGAGACG | | |
| | ACAT | | |
| CES1-R | (Antisense) | | |
| | ACTCCTGCTTGTTAATTCC | | |
| | GACC | | |
| CES2-F | (Sense) | NM_198061 | 63° C. |
| | AACCTGTCTGCCTGTGACC | | |
| | AAGT | | |
| CES2-R | (Antisense) | | |
| | ACATCAGCAGCGTTAACAT | | |
| | TTTCTG | | |
| ID4-EX-F | (Sense) | BC014941 | 50° C. |
| | CCTGCAGCACGTTATCG | | |
| | ACT | | |
| ID4-EX-R | (Antisense) | | |
| | CTCAGCGGCACAGAATGC | | |

F: forward primer;
R: reverse primer;
SP: sequencing primer.

### Pyrosequencing for Methylation Analysis

Bisulfite-converted DNA was used for pyrosequencing analysis as previously described (29). In brief, PCR product of each gene was used for individual sequencing reaction. Streptavidin-Sepharose beads (Amersham Biosciences, Uppsala, Sweden) and Vacuum Prep Tool (Biotage AB, Uppsala, Sweden) was used to purify the single-stranded biotinylated PCR product per the manufacturer's recommendation. The appropriate sequencing primer was annealed to the purified PCR product and used for a pyrosequencing reaction using the PSQ 96HS system (Biotage AB, Uppsala, Sweden). Raw data were analyzed with the allele quantification algorithm using the provided software. Pyrosequencing was done for LINE-1 elements and ID4 tumor suppressor gene. The PCR primers used are indicated in Table 1.

### Inhibition of Carboxylesterase by NDGA

For dose effects of nordihydroguaiaretic acid (NDGA) on CES inhibition, HepG2 cells were grown in 6-well plates. Cells were allowed to adhere overnight and were then treated with 100 μM of NDGA and 100 μM of NPEOC-DAC or 5 μM of DAC as indicated. Cells were harvested on day 4 by Tripsin-EDTA.

### Statistics

Microsoft Excel was used, to perform a standard two-sided t-test to calculate p values.

### RESULTS

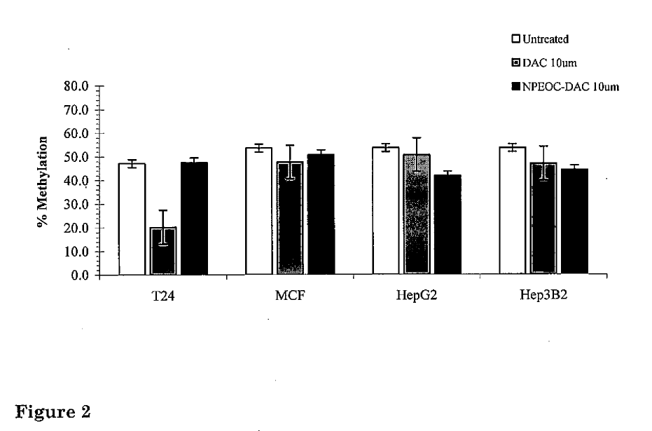

Four cancer cell lines T24 (bladder), MCF7 (breast), HepG2 (liver), and Hep3B (liver) were treated with DAC and NPEOC-DAC for three days. Global DNA methylation was quantified using bisulfite-PCR pyrosequencing of LINE-1 DNA repetitive elements (FIG. 2). PCR primers were designed towards a consensus LINE-1 sequence and a pool of LINE-1 elements was assayed as a surrogate of global DNA methylation. DAC decreased global DNA methylation by 57.1%, 11.0%, 5.6% and 12.3% in T24, MCF, HepG2 and Hep3B cell lines respectively. NPEOC-DAC decreased global DNA methylation by 0.9%, 5.1%, 22.1% and 17.4% in T24, MCF, HepG2 and Hep3B cell lines respectively. T24 cell lines were very sensitive to DAC, and only the liver cell lines showed a significant decrease in DNA methylation when treated with NPEOC-DAC (p=0.01).

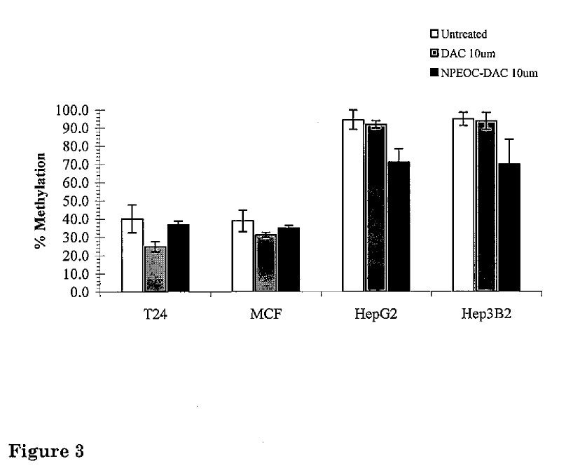

Gene specific DNA methylation changes were also assessed by measuring changes in ID4 (FIG. 3). Baseline methylation of ID4 was 40.2%, 39.0%, 94.7% and 95.4% in the T24, MCF, HepG2 and Hep3B cell lines respectively. There was a higher baseline methylation of ID4 in the liver cell lines. DNA methylation of ID4 decreased by 38.1%, 19.7%, 2.6% and 1.1% in T24, MCF, HepG2 and Hep3B cell lines respectively when treated with DAC. Conversely, DNA methylation of ID4 decreased by 8.2%, 10.0%, 24.7% and 26.2% in T24, MCF, HepG2 and Hep3B cell lines respectively when treated with NPEOC-DAC. DAC decreased ID4 methylation very efficiently in T24 cells, but the other cell lines seemed more resistant to DAC at 10 uM. Again NPEOC-DAC significantly decreased DNA methylation only in HepG2 and Hep3B cell lines (P=0.0005).

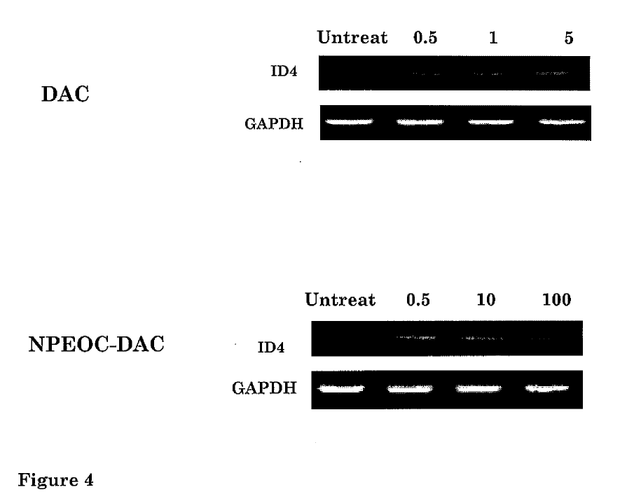

After verifying that NPEOC-DAC was able to inhibit the hypermethylation of ID4, the inventors next examined whether NPEOC-DAC could reactivate expression of this epigenetically silenced gene. HepG2 cells do not normally express ID4, however when treated with decitabine for 3 days clear expression of ID4 could be detected by reverse transcription PCR (FIG. 4). Treatment of HepG2 cells with NPEOC-DAC also clearly showed increased expression of ID4. Therefore both decitabine and NPEOC-DAC clearly decreased the methylation of the ID4 promoter, which led to increased expression of ID4 mRNA.

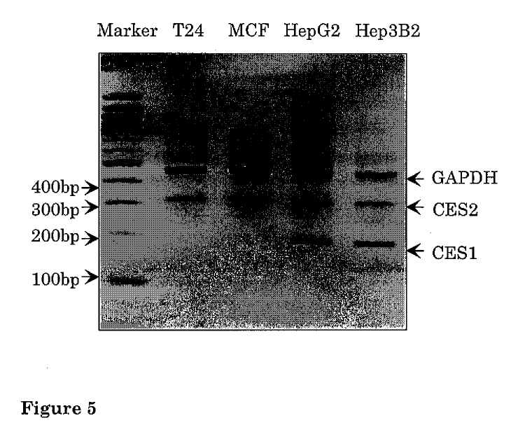

While not wanting to be bound by the theory, the inventor believes that the enzyme carboxylesterase is responsible for the cleavage of the N4 carboxylester bond needed to convert NPEOC-DAC into DAC. The inventor wanted to assess carboxylesterase expression in the cell lines studied. Multiplex RT-PCR assays were used to determine expression of carboxylesterase 1 (CES1) and 2 (CES2) in the cell lines studied. CES1 was expressed only in the liver cells HepG2 and Hep3B. CES2 was more broadly expressed in all the cell lines, but at lower levels (FIG. 5).

T24 and HepG2 cells were used for more detailed investigations of the ability of NPEOC-DAC to inhibit DNA methylation. Untreated HepG2 cells have a baseline LINE-1 methylation of 53.9%. This decreased slightly when treated with 0.5 μM, 1 μM or 5 μM DAC to 46.0%, 47.0% and 56.9% respectively. This paradoxical increase at higher concentrations is associated with significant cell toxicity, which is believed to be selecting for cells with higher methylation levels. T24 cells showed a lower baseline LINE-1 methylation of 47.3%, but were more sensitive to DAC. LINE-1 methylation decreased to 26.8%, 21.0% and 35.7% when treated with 0.5 uM, 1 uM and 5 uM DAC. As previously described there was a parabolic effect on the effect of DNA methylation with lower doses around 1 uM having the maximal effect on DNA methylation.

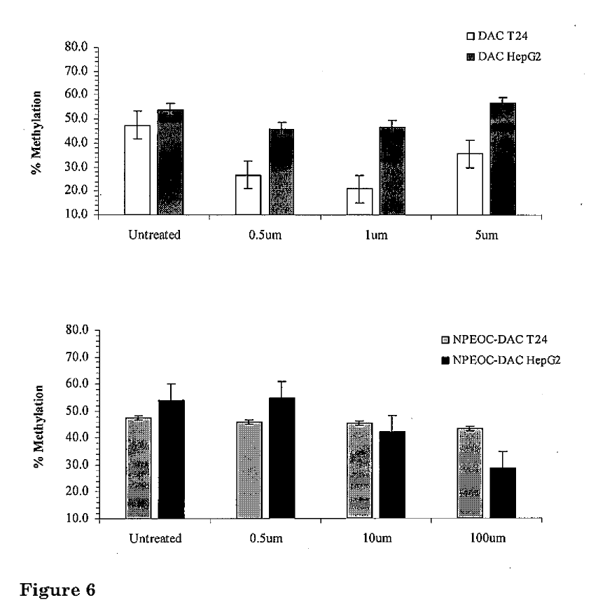

NPEOC-DAC effectively inhibited DNA methylation in HepG2 cells, but required higher concentrations. NPEOC-DAC at 0.5 μM had no detectable effect on DNA methylation, but 10 μM NPEOC-DAC decreased methylation to 42.0% and 100 μM NPEOC-DAC was able to decrease LINE-1 methylation to 28.7%. This decrease was greater than the strongest effect observed with DAC. T24 cells were unaffected by NPEOC-DAC, and there appeared to be no change in DNA methylation even at the highest concentrations of NPEOC-DAC (FIG. 6).

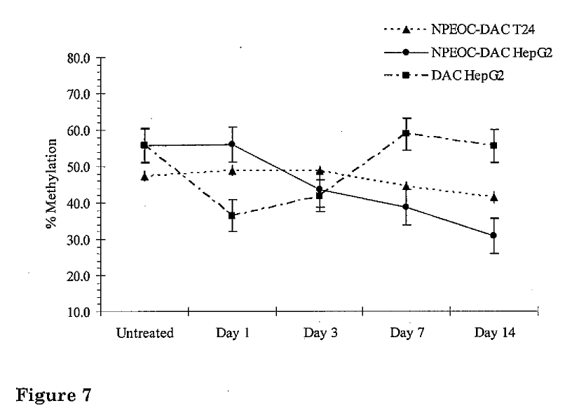

NPEOC-DAC is a prodrug and needs to be converted to DAC in order to have activity. The time dependence of NPEOC-DAC to inhibit DNA methylation was therefore investigated. DAC significantly decreased methylation in HepG2 cells from 53.9% to 36.5% by day 1 of treatment. However, subsequent days of treatment led to a decrease in this effect, and by day 7 the LINE-1 methylation returned to baseline by day 7. Again, this treatment with high concentrations of DAC, +10 uM, was associated with significant toxicity. Thus, the inventor believes there must be a selection phenomenon where the cells with a selective advantage have higher LINE-1 methylation levels. Conversely, NPEOC-DAC did not begin to decrease LINE-1 methylation until day 3 of treatment. LINE-1 methylation decreased from 53.9% to 43.7% on day 3, and to 38.9% on day 7 and finally 30.9% on day 14. This delayed effect could be attributable to the time required to convert NPEOC-DAC to DAC. In addition, there was less toxicity than observed with DAC, which is believed to be due to the slow conversion of NPEOC-DAC to DAC. T24 cells did not show any decrease in DNA methylation despite prolonged treatment with NPEOC-DAC (FIG. 7).

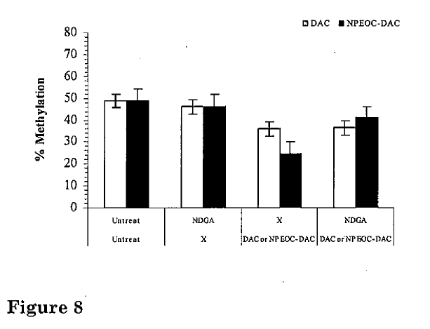

Nordihydroguaiaretic acid (NDGA) is a known inhibitor of carboxylesterase (30). In order to confirm the ability of NPEOC-DAC to inhibit DNA methylation is dependent on carboxylesterase, the inventor treated cells with a combination of NDGA and NPEOC-DAC. NDGA at 100 μM had no effect on the DNA methylation of HepG2 cells. HepG2 cells treated with 10 μM NPEOC-DAC alone decreased LINE-1 methylation by 49.7% to 24.5%. However, NPEOC-DAC in combination with NDGA showed that LINE-1 methylation did not decrease. Thus, NDGA inhibited the ability of NPEOC-DAC to inhibit DNA methylation (FIG. 8).

It was discovered that NPEOC-DAC could inhibit DNA methylation in cells expressing CES1 but not cells expressing CES2, and that inhibition of CES by NDGA blocked the ability of NPEOC-DAC to inhibit DNA methylation. The dependence of NPEOC-DAC on CES1 was further demonstrated by carrying out transient transfection of CES1 or CES2 along with NPEOC-DAC treatment in an epigenetic GFP reported system. NIH3T3 cells with a stably transfected GFP reporter gene were cultured until the GFP expression became epigenetically extinguished, and then a clone that could be reactivated by 5-aza-2′-deoxyazacytidine was selected. This cell line was then treated with NPEOC-DAC without reactivation of GFP. However, transient transfection of the human CES1 gene and treatment with NPEOC-DAC lead to reactivation of GFP. Transfection of CES1 alone without NPEOC-DAC treatment did not reactivate GFP. Transfection of CES2 had no effect on NPEOC-DAC treatment and activation of GFP. NPEOC-DAC activation is dependent on CES1 expression.

The present invention demonstrates that NPEOC-DAC can inhibit global DNA methylation as shown by the induction of hypomethylation of the LINE-1 repetitive element. Furthermore, the present invention demonstrates that NPEOC-DAC can reverse hypermethylation of a tumor suppressor gene, ID4, and reactivate expression of this gene. This ability to inhibit DNA methylation is specific for the liver cancer cell lines HepG2 and Hep3B, and is dependent on the activity of the carboxylesterase 1 enzyme. Thus, NPEOC-DAC is a prodrug that can be metabolized to 5-aza-2′-deoxyazacytidine and incorporated into DNA. This, in turns, leads to irreversible inhibition of the DNA methyltransferase enzyme.

Unlike decitabine, NPEOC-DAC was less potent at inhibiting DNA methylation. NPEOC-DAC was 23-fold less efficient at inhibiting DNA methylation compared to DAC. In addition there appeared to be a 3 day delay in the effects of NPEOC-DAC compared to DAC. While not wanting to be bound by the theory, the inventors believe that this is due to the delayed effect of converting the prodrug NPEOC-DAC to DAC. Capecitabine, which is activated to 5-FU by a similar mechanism, appears to be much more efficiently converted to its active form. The inventors believe that the N4 protecting group of capecitabine with a simple 5-carbon chain at the N4 position allows for more efficient cleavage of capecitabine to 5-fluorocytidine. Thus, changing the N4 NPEOC group of NPEOC-DAC to a smaller carbon chain should lead to a molecule much more efficient at inhibiting DNA methylation.

### REFERENCES

1. Bird A. The essentials of DNA methylation. Cell 1992; 70(1):5-8.
2. Lu S C. S-Adenosylmethionine. Int J Biochem Cell Biol 2000; 32(4):391-5.
3. Jones P A, Buckley J D. The role of DNA methylation in cancer. Adv Cancer Res 1990; 54:1-23.
4. Spruck C H, 3rd, Rideout W M, 3rd, Jones P A. DNA methylation and cancer. Exs 1993; 64:487-509.
5. Issa J P. CpG island methylator phenotype in cancer. Nat Rev Cancer 2004; 4(12):988-93.
6. Jones P A, Taylor S M. Cellular differentiation, cytidine analogs and DNA methylation. Cell 1980; 20(1):85-93.
7. Sheikhnejad G, Brank A, Christman J K, et al. Mechanism of inhibition of DNA (cytosine C5)-methyltransferases by oligodeoxyribonucleotides containing 5,6-dihydro-5-azacytosine. J Mol Biol 1999; 285(5):2021-34.
8. Randerath K, Tseng W C, Harris J S, Lu L J. Specific effects of 5-fluoropyrimidines and 5-azapyrimidines on modification of the 5 position of pyrimidines, in particular the synthesis of 5-methyluracil and 5-methylcytosine in nucleic acids. Recent Results Cancer Res 1983; 84:283-97.
9. Marquez V E, Kelley J A, Agbaria R, et al. Zebularine: a unique molecule for an epigenetically based strategy in cancer chemotherapy. Ann N Y Acad Sci 2005; 1058:246-54.
10. Taylor S M, Jones P A. Multiple new phenotypes induced in 10T1/2 and 3T3 cells treated with 5-azacytidine. Cell 1979; 17(4):771-9.
11. Grant S, Bhalla K, Gleyzer M. Interaction of deoxycytidine and deoxycytidine analogs in normal and leukemic human myeloid progenitor cells. Leuk Res 1986; 10(9):1139-46.
12. Issa J P, Garcia-Manero G, Giles F J, et al. Phase 1 study of low-dose prolonged exposure schedules of the hypomethylating agent 5-aza-2′-deoxycytidine (decitabine) in hematopoietic malignancies. Blood 2004; 103(5): 1635-40.
13. Yang A S, Doshi K D, Choi S W, et al. DNA methylation changes after 5-aza-2′-deoxycytidine therapy in patients with leukemia. Cancer Res 2006; 66(10):5495-503.
14. Davidson S, Crowther P, Radley J, Woodcock D. Cytotoxicity of 5-aza-2′-deoxycytidine in a mammalian cell system. Eur J Cancer 1992; 28(2-3):362-8.
15. Notari R E, DeYoung J L. Kinetics and mechanisms of degradation of the antileukemic agent 5-azacytidine in aqueous solutions. J Pharm Sci 1975; 64(7):1148-57.
16. Rudek M A, Zhao M, He P, et al. Pharmacokinetics of 5-azacitidine administered with phenylbutyrate in patients with refractory solid tumors or hematologic malignancies. J Clin Oncol 2005; 23(17):3906-11.
17. Momparler R L. Pharmacology of 5-Aza-2′-deoxycytidine (decitabine). Semin Hematol 2005; 42(3 Suppl 2):S9-16.
18. Kaminskas E, Farrell A, Abraham S, et al. Approval summary: azacitidine for treatment of myelodysplastic syndrome subtypes. Clin Cancer Res 2005; 11(10):3604-8.
19. van Groeningen C J, Leyva A, O'Brien A M, Gall H E, Pinedo H M. Phase I and pharmacokinetic study of 5-aza-2′-deoxycytidine (NSC 127716) in cancer patients. Cancer Res 1986; 46(9):4831-6.
20. Cheng J C, Matsen C B, Gonzales F A, et al. Inhibition of DNA methylation and reactivation of silenced genes by zebularine. J Natl Cancer Inst 2003; 95(5):399-409.
21. Gabbara S, Bhagwat A S. The mechanism of inhibition of DNA (cytosine-5-)-methyltransferases by 5-azacytosine is likely to involve methyl transfer to the inhibitor. Biochem J 1995; 307 (Pt 1):87-92.
22. Miwa M, Ura M, Nishida M, et al. Design of a novel oral fluoropyrimidine carbamate, capecitabine, which generates 5-fluorouracil selectively in tumours by enzymes concentrated in human liver and cancer tissue. Eur J Cancer 1998; 34(8):1274-81.
23. Verweij J. Rational design of new tumoractivated cytotoxic agents. Oncology 1999; 57 Suppl 1:9-15.
24. Moertel C G, Rubin J, O'Connell M J, Schutt A J, Wieand H S. A phase II study of combined 5-fluorouracil, doxorubicin, and cisplatin in the treatment of advanced upper gastrointestinal adenocarcinomas. J Clin Oncol 1986; 4(7):1053-7.
25. Tominaga T, Nomura Y, Uchino J, et al. Cyclophosphamide, adriamycin, 5-fluorouracil and high-dose toremifene for patients with advanced/recurrent breast cancer. The Japan Toremifene Cooperative Study Group. Jpn J Clin Oncol 1998; 28(4):250-4.
26. Wolff R K, Frazer K A, Jackler R K, Lanser M J, Pitts L H, Cox D R. Analysis of chromosome 22 deletions in neurofibromatosis type 2-related tumors. Am J Hum Genet 1992; 51(3):478-85.
27. Gonzalez-Zulueta M, Bender C M, Yang A S, et al. Methylation of the 5′ CpG island of the p16/CDKN2 tumor suppressor gene in normal and transformed human tissues correlates with gene silencing. Cancer Res 1995; 55(20):4531-5.
28. Clark S J, Harrison J, Paul C L, Frommer M. High sensitivity mapping of methylated cytosines. Nucleic Acids Res 1994; 22(15):2990-7.
29. Yang A S, Estecio M R, Doshi K, Kondo Y, Tajara E H, Issa J P. A simple method for estimating global DNA methylation using bisulfite PCR of repetitive DNA elements. Nucleic Acids Res 2004; 32(3):e38.
30. Schegg K M, Welch W, Jr. The effect of nordihydroguaiaretic acid and related lignans on formyltetrahydrofolate synthetase and carboxylesterase. Biochim Biophys Acta 1984; 788(2):167-80.

The entire disclosure of each publication cited herein is incorporated by reference.

TABLE 1

Primers used in quantitative pyrosequencing
assays and RT-PCR

| | | | |
|---|---|---|---|
| | | | Optimal |
| | | GenBank | annealing |
| Primer | Sequence (5′ to 3′) | accession # | temperature |

For bisulfite pyrosequencing

| | | | |
|---|---|---|---|
| LINE-F | (Sense) | X58075 | 53° C. |
| | TTTTGAGTTAGGTGTGGGA | | |
| | TATA | | |
| | (SEQ ID NO:1) | | |
| LINE-R | (Antisense)biotin- | | |
| | AAAATCAAAAAATTCCCTT | | |
| | TC | | |
| | (SEQ ID NO:2) | | |
| LINE-SP | (Sense) | | |
| | AGTTAGGTGTGGGATAT | | |
| | AGT | | |
| | (SEQ ID NO:3) | | |
| ID4-F | (Sense) | BC014941 | 55° C. |
| | TTTGATTGGTTGGTTATTT | | |
| | TAGA | | |
| | (SEQ ID NO:4) | | |
| ID4-R | (Antisense) biotin- | | |
| | AATATCCTAATCACTCCCT | | |
| | TC | | |
| | (SEQ ID NO:5) | | |
| ID4-SP | (Sense) | | |
| | GGTTTTATAAATATAGTTG | | |
| | (SEQ ID NO:6) | | |

For expression

| | | | |
|---|---|---|---|
| GAPDH-F | (Sense) | AB062273 | 63° C. |
| | TGAGGCTGTTGTCATACTT | | |
| | CTC | | |
| | (SEQ ID NO:7) | | |
| GAPDH-R | (Antisense) | | |
| | CAGCCGAGCCACATCG | | |
| | (SEQ ID NO:8) | | |
| CES1-F | (Sense) | AB119996 | 63° C. |
| | AGAGGAGCTCTTGGAGACG | | |
| | ACAT | | |
| | (SEQ ID NO:9) | | |
| CES1-R | (Antisense) | | |
| | ACTCCTGCTTGTTAATTCC | | |
| | GACC | | |
| | (SEQ ID NO:10) | | |
| CES2-F | (Sense) | NM_198061 | 63° C. |
| | AACCTGTCTGCCTGTGACC | | |
| | AAGT | | |
| | (SEQ ID NO:11) | | |
| CES2-R | (Antisense) | | |
| | ACATCAGCAGCGTTAACAT | | |
| | TTTCTG | | |
| | (SEQ ID NO:12) | | |
| ID4-EX-F | (Sense) | BC014941 | 50° C. |
| | CCTGCAGCACGTTATCG | | |
| | ACT | | |
| | (SEQ ID NO:13) | | |
| ID4-EX-R | (Antisense) | | |
| | CTCAGCGGCACAGAATGC | | |
| | (SEQ ID NO:14) | | |

F: forward primer;
R: reverse primer;
SP: sequencing primer.

What is claimed is:

1. A method of reducing nucleic acid methylation in a cell, comprising:
identifying a cell in which the level of nucleic acid methylation is higher than a control level; and
contacting the cell with a compound in which an epigenetic agent, a cell targeting agent, or a carbon chain or ring containing 2-10 carbons is attached to a pyrimidine analog that inhibits nucleic acid methylation through an N4 carboxylester or ester bond, thereby reducing nucleic acid methylation in the cell.

2. The method of claim 1, wherein the pyrimidine analog is 5-azacytidine, 5-aza-2′-deoxycytidine, or 5-fluorocytidine.

3. The method of claim 1, wherein the epigenetic agent is a histone deacetylase inhibitor, histone methyltransferase inhibitor, methyl-binding protein inhibitor, or SIRT (sirtuin class of human protein deacetylases) inhibitor.

4. The method of claim 1, wherein the compound is 2′-deoxy-N4-[2-(4-nitrophenyl)ethoxycarbonyl]-5-azacytidine (NPEOC-DAC).

5. The method of claim 4, wherein the cell expresses carboxylesterase 1 (CES1).

6. The method of claim 4, wherein the cell is a liver cancer cell.

7. The method of claim 1, wherein the cell-targeting agent is acetylcholine.

8. The method of claim 7, wherein the cell expresses acetylcholinesterase.

9. The method of claim 7, wherein the cell is a cell of the central nervous system.

10. A composition comprising a pharmaceutically acceptable carrier and a compound in which an epigenetic agent, a cell targeting agent, or a carbon chain or ring containing 2-10 carbons is attached to a pyrimidine analog that inhibits nucleic acid methylation through an N4 carboxylester or ester bond.

11. The composition of claim 10, wherein the pyrimidine analog is 5-azacytidine, 5-aza-2′-deoxycytidine, or 5-fluorocytidine.

12. The composition of claim 11, wherein the compound is NPEOC-DAC.

13. The composition of claim 10, wherein the pharmaceutically acceptable carrier is a hydrophobic solvent, a polar solvent, or a hydrophobic-polar solvent.

14. The composition of claim 10, wherein the pharmaceutically acceptable carrier is dimethyl sulfoxide (DMSO).

15. A compound, comprising a therapeutic agent or a cell-targeting agent attached to a pyrimidine analog through an N4 carboxylester or ester bond.

16. The compound of claim 15, wherein the therapeutic agent is an epigenetic agent.

17. The compound of claim 15, wherein the pyrimidine analog is 5-azacytidine, 5-aza-2′-deoxycytidine, 5-fluorocytidine, or cytosine arabinoside.

18. The compound of claim 17, wherein the compound has the following structure:

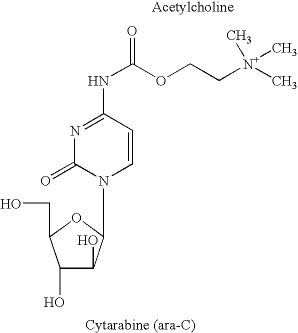

19. The compound of claim 17, wherein the compound has the following structure:

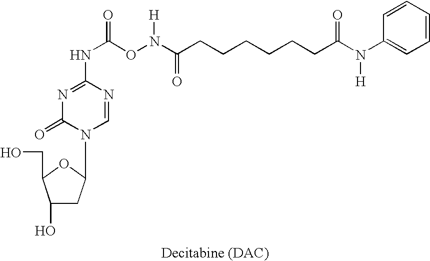

20. The compound of claim 17, wherein the compound has the following structure:

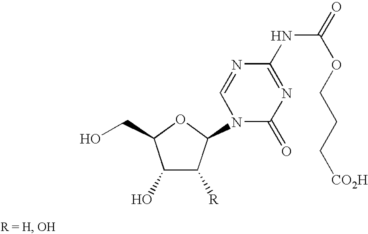

21. The compound of claim 17, wherein the compound has the following structure:

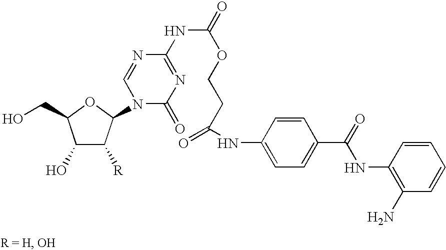

22. The compound of claim 17, wherein the compound has the following structure:

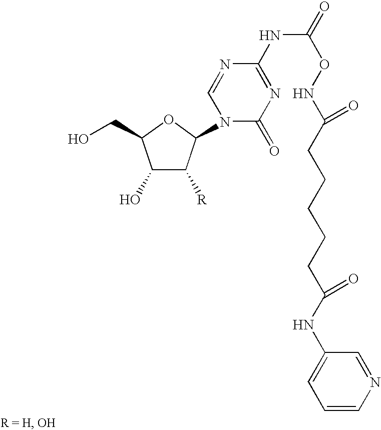

23. The compound of claim 17, wherein the compound has the following structure:

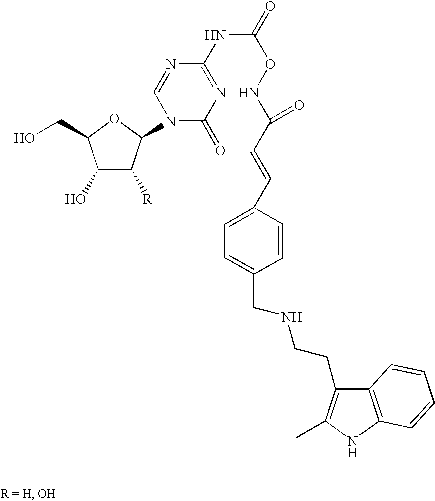

24. A method of modulating the biological activity of a cell, comprising contacting a cell with a compound of claim 15, thereby modulating the biological activity of the cell.

25. The method of claim 23, wherein the cell is a cancer cell.
